(19) United States
(12) Patent Application Publication
(10) Pub. No.: US 2010/0137676 A1
(43) Pub. Date: Jun. 3, 2010

(54) N-ACETYLCYSTEINE AMIDE (NAC AMIDE) FOR TREATMENT OF OXIDATIVE STRESS ASSOCIATED WITH INFERTILITY

(76) Inventor: Glenn A. Goldstein, New York, NY (US)

Correspondence Address: MINTZ, LEVIN, COHN, FERRIS, GLOVSKY AND POPEO, P.C, ONE FINANCIAL CENTER, BOSTON, MA, 02111, US

(21) Appl. No.: 12/696,399

(22) Filed: Jan. 29, 2010

Related U.S. Application Data
(63) Continuation of application No. 11/912,294, filed on Nov. 8, 2007, now abandoned.
(60) Provisional application No. 60/673,559, filed on Apr. 21, 2005.

(51) Int. Cl. A61B 17/425; C12N 5/02; A01N 1/02; A61D 19/00

(52) U.S. Cl. 600/35; 435/404; 435/2

(57) ABSTRACT

An in vitro culture and/or fertilization medium containing N-acetylcysteine amide (NAC amide) reduces or prevents oxidative stress and free radical formation that contribute to the cellular damage and eventual demise of sperm, oocytes and embryos that are cultured, fertilized and maintained in vitro. The NAC amide-containing medium composition for in vitro culture and fertilization is suitable for use in the culture of oocytes, in the culture and development of early embryos, in the preparation or culture of sperm, and in the pre-treatment of oocytes or sperm. The NAC amide-containing composition supports the growth of viable embryos until blastocyst stage.

### FIELD OF THE INVENTION

The present invention generally relates to the use of antioxidants in reducing oxidative stress that leads to decreased oocyte quality, fertilization and embryo viability to promote in vivo and in vitro survival and improved function of sperm, oocytes, and embryos.

### BACKGROUND OF THE INVENTION

In nature, fertilization occurs by sperm cells being deposited into the female of warm-blooded animal species (including humans) and then binding to and fusing with an oocyte. This fertilized oocyte then divides to form an embryo. Over the last several decades, the use of assisted reproduction techniques has allowed scientists and clinicians to intervene in these events to treat poor fertility in some individuals, or to store sperm, oocytes or embryos for use at other locations or times.

The procedures utilized in cases of assisted reproduction include washing a sperm sample to separate out the sperm-rich fraction from non-sperm components, such as seminal plasma or debris; further isolating the healthy, motile (swimming) sperm from dead sperm or from white blood cells in an ejaculate; freezing or refrigerating the sperm (storage) for use at a later date or for shipping to females at differing locations; extending or diluting sperm for culture in diagnostic testing or for use in therapeutic interventions such as in vitro fertilization (IVF) or intracytoplasmic sperm injection (ICSI); culturing or freezing of oocytes from the female for use in in vitro fertilization; and culturing or freezing of embryos prior to implanting into a female in order to establish a pregnancy.

At each step of the way, in vitro intervention decreases the normal survival and function of sperm, oocytes, and embryos. Much research has been dedicated toward improving these procedures; however, overall success remains limited. For example, less than 20% of IVF attempts result in the birth of a child. In addition, only half or fewer sperm cells routinely survive the freezing process, such that pregnancy rates with frozen sperm from donors average between 10 and 20%. Oocytes and embryos also show significantly disrupted function after culture or freezing. Specifically, human oocytes survive the freezing process at very low levels. Thus, in spite of several decades of work, much room remains for improvement in the field of assisted reproduction technologies, especially in gamete and embryo handling, culture, and storage.

One common procedure used in sperm collection is washing sperm cells. Washing sperm cells prior to use in assisted reproduction technologies is important for a variety of reasons. Seminal plasma contains, in addition to sperm cells, sugars and proteins that can be toxic to the sperm cells in a sample. Also, sperm samples that have been frozen contain cryopreservation media that needs to be washed from the sperm cells prior to insemination in the female of some species, particularly in birds and humans. For all species, cryopreservative media cause lipid membrane peroxidation (LPO) and degeneration of the sperm after thawing. Generally, washing involves centrifuging a sample of semen or thawed sperm through a diluting wash media, which allows collection of a sperm-rich pellet. Although a very common procedure, centrifugation itself can cause sperm lipid peroxidation and membrane breakdown.

After a sperm wash process, or in place of it, a specific procedure for the isolation of the motile sperm from a sample may be done. A sperm sample contains dead and dying sperm that release enzymes that can damage the live, motile sperm. In addition, the sample contains white blood cells, red blood cells, and bacteria which are also toxic to the healthy sperm. Sperm isolations involve separating out the live, healthy, and motile sperm for use in diagnostic or therapeutic procedures. Generally, sperm are isolated by allowing the motile sperm to swim away from the dead sperm and debris (sperm swim-up), by centrifuging the sperm through a density gradient, or by passing the sperm through a column that binds the dead sperm and debris. Each of these techniques has its own disadvantages. Swim-up only recovers low sperm numbers, and it requires a long culture period. Current centrifugation gradient reagents are generally toxic to sperm, such that an added wash step is necessary to remove the gradient solution from the sperm sample. Column methods have poor selectivity for motile sperm and do not always result in good recovery of sperm numbers from a full ejaculate.

Once sperm have been washed or isolated, they are then extended (or diluted) in culture or holding media for a variety of uses. Existing sperm culture techniques result in losses of motile sperm and also damage sperm DNA over time in culture. Although sperm survive for days in the females of most species, sperm survival in culture is typically only half as long as that seen in vivo. Poor quality sperm may survive for even shorter time periods in culture. Much of this damage is due to lipid peroxidation of the membrane and DNA or to chromatin breakdown. Sperm are extended in media for use in sperm analysis and diagnostic tests; assisted reproduction technologies, such as IVF, gamete intrafallopian transfer, insemination into the female, ICSI; and holding prior to cryopreservation. Each of these uses for extended or diluted sperm requires a somewhat different formulation of basal medium; however, in all cases sperm survival is suboptimal outside of the female reproductive tract.

Likewise, oocytes and embryos often develop abnormally (e.g., chromosome number, cytoskeleton formation) in culture, compared with in vivo conditions. Additionally, current culture methods utilize high doses of animal proteins, for example, serum, which may result in an oversized fetus and perinatal complications for the offspring.

Co-culturing sperm, oocytes and embryos with cell feeder layers, can overcome some of the difficulties in assisted reproduction technologies. However, co-cultures are of variable quality and variable reliability and add the risk of pathogen transfer from the feeder cells to the gametes or embryos that are to be transferred back to living animals or humans

The storage of sperm, oocytes and embryos is of widespread importance in commercial animal breeding programs, human fertilization and sperm donor programs and in dealing with some disease states. For example, sperm samples may be frozen for men who have been diagnosed with cancer or other diseases that may eventually interfere with sperm production. Freezing and storage of sperm is critical in the area of preservation of endangered species. Many of these species have semen, which does not freeze well under existing methods. In standard animal husbandry, artificial insemination (AI) with frozen bull sperm is used in 85% of dairy cows. Because most commercial turkeys have become too heavy to mate naturally, AI is required on almost all turkey farms. Approximately six million turkey hens are inseminated each week in the United States. However, existing methods of storing collected turkey sperm cannot support sperm survival for even the several hours required to transport semen between farms, much less for long-term freezing. This limits the ability to store or transport genetic material to improve production. Human donor AI is also used for couples with severe male infertility; however, the rate of pregnancy using donor semen is only a quarter of that occurring with natural reproduction. Furthermore, surgical insemination may be required.

Current techniques for freezing sperm from all species result in membrane damage and subsequent death of about half of the sperm cells in a sample. Much of this damage occurs by reactive oxygen species causing lipid peroxidation of the sperm membrane. Despite these widespread and serious problems, the state of the art and protocols for this field have changed very little in the last 15 years. In view of the increasing use of frozen sperm for a variety of needs, new methods and conditions for culturing, freezing, or storing sperm would offer advantages for animal producers, as well as human fertility specialists.

Freezing oocytes and embryos is also important for preserving genetic material from endangered species, increasing offspring production from valuable livestock, or for retaining embryos for infertile couples prior to transfer. Current methods of freezing oocytes and embryos are less than optimal and decreased development potential is typical. In fact, human oocytes are rarely successfully frozen, thus requiring the implantation of multiple embryos into a woman's uterus, which increases the number of dangerous and high risk, multiple pregnancies. In addition, IVF embryos or genetically altered embryos from all species, such as those obtained after gene therapy, have very poor post-freezing survival rates with existing freezing media. This includes cloned embryos and embryos derived from embryonic stem cells (ESC).

In vitro fertilization and embryo transfer involve the fertilization of oocytes and sperm in vitro and then transplanting the developed embryos into a female body. Since the first report of a human birth following in vitro fertilization in England in 1978 by Edwards et al., and due to recent progress in the developmental technology, this procedure has been rapidly and widely used throughout the world. In Japan, for example, in vitro fertilization is now an indispensable treatment for sterility. In spite of recent advances in in vitro fertilization techniques and procedures, only a few cases actually lead to pregnancy. Although one cause may be due to lower fertility in sterile male patients, the lower implantation rate of transplanted oocytes seems to be a main cause. (Mori, Munehide et al., Nippon sankahujin kagakukai zashi, 45:397, (1993); Cohen, J. et al., VIIIth World Congress on in vitro Fertilization and Alternate Assisted Reproduction Kyoto, Sep., 12-15 (1993), World Collaborative Report (1991)).

In addition to technical factors, a decreased quality of embryos during culture seems to be responsible for such lower implantation rates. (Inoue, Masahito, Rinsho fujinka sanka, 48:148, (1994)). Because mammalian oocytes do not have substances that correspond to the albumin in the eggs of reptiles and birds, the amounts of nutrients reserved in oocytes are naturally low. Thus, in the early-stage embryos of in vitro fertilization, nutrients from the culture medium must be taken up through the zona pellucida. Chemically defined media such as Ham's F-10 medium, MEM (Minimum Essential Medium), Dulbecco's MEM and the like, which have been conventionally utilized in in vitro fertilization techniques, were not originally developed to support in vitro fertilization. However, these media, or modified counterparts, are conventionally used in tissue culture; thus they are not necessarily optimal for the nutrient requirements of early embryos cultured in vitro.

Human Tubal Fluid (HTF) Medium has been developed as a suitable nutrient-containing medium for human in vitro fertilization. HTF medium comprises a composition that approximates the electrolyte composition of human oviduct fluid (Quinn, P. J. et al., Fertility and Sterility, 44:493 (1982)). This medium is commercially available and typically replaces Ham's F-10 medium that was previously used. However, because the HTF medium only contains electrolytes as the main components and glucose as an energy source, this medium shows no improvement over the Ham's F-10 medium containing amino acids in terms of nutrient composition. In fact, despite the use of HTF medium, the embryo implantation rate is not enhanced and an amelioration of embryo quality remains unimproved.

In order to compensate for this disadvantage, a method has been utilized in which cultured embryos are maintained by adding to the medium female serum that has been inactivated by heat treatment. The serum contains growth factors and the like, in addition to proteins, carbohydrates, lipids, vitamins and minerals as nutrients which are essential factors in animal cell culture. However, it has been reported that such serum is not always needed in the in vitro fertilization-embryo transfer process (Menezo, Y. et al., Fertility and Sterility, 42:750 (1984)). Indeed, the growth of embryos may even be suppressed by the addition of female serum (Mehita et al., Biology of Reproduction, 43:600 (1990)). Further, because the serum itself is difficult to collect and there is a danger of contamination by viruses etc., female serum is not suitable for use as an additive for the medium of in vitro fertilized oocytes.

Free radicals have been reported to have significant growth-suppressing effects on embryos. This is based on the theory that the growth of cultured embryos is suppressed by oxidative stress, which causes more direct contact of cells with oxygen in vitro, compared with in vivo (Whitten, W., Advanced in the Biosciences, 6:129 (1971); Quinn, P. J. et al., Journal of Experimental Zoology, 206:73 (1978)). Thus, the prevention of oxidative stress may enhance the growth of embryos. Certain components, such as superoxide dismutase (SOD), edetic acid (EDTA) and the like have been added to culture media in an attempt to conquer the effects of oxidative stress. (Abramczuk, J. et al., Developmental Biology, 61:378 (1977); Nonozaki, T. et al., Journal of Assisted Reproduction and Genetics, v9:274 (1992)).

In addition, it has also been reported that co-cultures using the epithelial cells of the oviduct, whose effective components are unknown, are effective for the growth of embryos (Xu, K. P. et al., Journal of Reproduction and Fertility, 94:33 (1992)) and that a growth factor such as insulin-like growth factor directly stimulates the growth of embryos (Matui, Motozumi et al., Honyudoubutu ranshi gakkaishi, 11:132 (19949). However, it has also been reported that such a co-culture is, at most, effective for the detoxification of a medium and there is no evidence available that the embryos obtain proper nutrients (Bavister, B. D., Human Reproduction, 7:1339 (1992)). In any event, most conventional media for in vitro fertilization and methods for the addition of additives to existing media, including the addition of superoxide dismutase, EDTA and the like, only partially prevent the cessation of the growth in vitro. Furthermore, the reported types of media are very inconvenient to handle because, during the actual culture of embryos, the optimal media allowing for the embryo's growth stages must be suitably selected and exchanged at every stage.

Accordingly, the demands of the field of in vitro fertilization are such that cultured oocytes, sperm and embryos require a culture medium and environment which are free of viral contaminants and contain nutrients and ingredients to maintain the viability and function of these cells for as long as possible under in vitro culture conditions. Such media should prevent damage to sperm and oocyte cells and to developing embryos by preventing or reducing oxidative stress and free radical formation in and around the cells in culture. The media should also be suitable for the treatment and/or pretreatment of sperm and oocytes, as well as for the growing early embryo during the in vitro fertilization-embryo transfer process. Ideally, the media is safe and can sustain all of the growth stages of the early embryo.

Needed in the art are new compounds and methods for safely supplementing incubation and culture media and fertility products to safeguard the viability of oocytes and sperm. Needed also are compounds and methods for use in culture media for in vitro fertilization to provide the appropriate conditions for the survival and maturation of oocytes and sperm, both prior to and following fertilization, and for the proper and healthy development of the resulting embryos.

### SUMMARY OF THE INVENTION

The present invention provides the use of the antioxidant N-acetylcysteine amide (NAC amide), or a physiologically acceptable derivative thereof, as a supplement for incubation and culture media during oocyte maturation and fertilization, and for incubation and culture media for embryo culture following in vitro fertilization and subsequent early stage pre-implantation embryo development. NAC amide is provided for use in methods and compositions for improving the viability and function of germ cells (sperm and oocytes), embryos and zygote formation, both in vivo and in vitro.

The present invention provides a composition, preparation, or formulation comprising NAC amide, or a physiologically acceptable salt or ester thereof, that is non-toxic to sperm, oocytes or embryos, and which additionally improves their function and survival during in vitro handling and manipulation. The NAC amide-containing composition improves sperm and oocyte function for use by couples trying to conceive naturally, as well as for use in a variety of assisted reproduction techniques in humans and animals. The present invention further provides other related advantages.

One aspect of the present invention provides a method for increasing the rate of fertilization of sperm and oocytes during in vitro fertilization techniques by including in or supplementing the culture medium with NAC amide, or a physiologically acceptable derivative or salt or ester thereof. Media supplementation with NAC amide is also provided for increasing the rate of fertility of mammalian embryos.

Another aspect of the present invention provides NAC amide for use as an ingredient in culture medium for egg and/or sperm maturation, fertilization between sperm and oocytes and embryo and zygote development. The presence of NAC amide in implantation culture medium, prior to embryo implantation, can increase the formation, survival and development of the embryo by decreasing free radical and oxidation damage that can occur during culture.

In another aspect related to the previous aspects, the present invention provides NAC amide used in conjunction with another component or factor, e.g., granulocyte-macrophage-colony stimulating factor (GM-CSF) for increasing the viability and success of embryo development to the blastocyst stage and beyond.

In another aspect, the present invention provides NAC amide for use in methods and compositions involved in the production and maintenance of transgenic animal embryos and eggs. In accordance with this aspect, NAC amide supplied to the eggs and embryos of transgenic animals will improve the rate of full development of transgenic organisms during in vitro culture, as well as in in vivo.

In yet another of its aspects, the present invention provides methods and compositions comprising NAC amide to nurture and support stem cell or other germ cell transplantation into animals, including humans, as well as to support cell growth and cloning in vitro and in vivo.

A further aspect of the present invention provides a physiologically or pharmaceutically acceptable composition or preparation comprising NAC amide for ingestion by a female following embryo implantation into the uterus to provide an antioxidant to prevent or reduce conditions of post-implantation oxidative stress.

Another aspect of the invention provides a physiologically or pharmaceutically acceptable composition or preparation comprising NAC amide for ingestion by a male to provide an antioxidant that allows for healthy sperm development to reduce the adverse affects of free radicals or oxidative stress on sperm production and development and overall fertility.

Yet another aspect of the present invention provides a pharmaceutically acceptable composition comprising NAC amide, or a physiologically acceptable derivative or salt or ester thereof, to prevent, reduce, counteract, or alleviate oxidative stress which is associated with infertility in animals, including humans.

In another aspect, the present invention provides a pharmaceutically acceptable composition comprising NAC amide, or a physiologically acceptable derivative or salt or ester thereof, to prevent, reduce, counteract, or alleviate oxidative stress resulting from excesses of heme oxygenase and bilirubin, which adversely affect the survival and development of preterm neonates.

In another aspect, the invention provides a non-spermicidal lubricant for increasing fertilization potential in animals. The lubricant comprises NAC amide, or a physiologically acceptable derivative or salt or ester thereof; and a non-spermicidal lubricious compound. The lubricious compound may comprise glycerine, methylcellulose, propylene glycol, plant oils, or petroleum jelly, or a combination of glycerine and petroleum jelly, or a combination of polyethylene oxide, sodium carboxypolymethylene and methylparaben. The lubricant may be used in vivo by administration or placement in a vagina prior to coitus or artificial insemination, or used during semen collection, such as by applying the lubricant to a male sexual organ prior to ejaculation into a receptacle or collecting sperm into a receptacle containing the lubricant. The lubricant may also be used to lubricate medical devices prior to reproductive procedures.

Additional aspects, features and advantages afforded by the present invention will be apparent from the detailed description and exemplification hereinbelow.

### DETAILED DESCRIPTION OF THE INVENTION

The present invention involves the use of an effective antioxidant, glutathione N-acetylcysteine amide (NAC amide), or a physiologically or pharmaceutically acceptable derivative or salt or ester thereof, as a supplement in culture medium composition for in vitro fertilization. Such a NAC amide-supplemented medium is particularly applied to the culture of oocytes, sperm, early embryos, which are fertilized oocytes, or to the pretreatment of oocytes or sperm prior to fertilization. A composition comprising NAC amide, e.g., water-soluble NAC amide can also be formulated and concentrated prior to adding to the medium according to the present invention. The concentrated formulation is diluted upon addition into the medium, or prior to addition to the medium. NAC amide, or a formulation containing NAC amide or its physiologically acceptable salt or ester, is effective for the stimulation of the growth and qualitative stabilization of early embryos and is suitable for the culture and successful development of early embryos in vitro.

Glutathione N-acetylcysteine amide (NAC amide), the amide form of N-acetylcysteine (NAC), is a novel low molecular weight thiol antioxidant and a Cu²⁺ chelator. NAC amide provides protective effects against cell damage in its role as a scavenger of free radicals. In mammalian red blood cells (RBCs), NAC amide has been shown to inhibit tert.-butylhydroxyperoxide (BuOOH)-induced intracellular oxidation and to retard BuOOH-induced thiol depletion and hemoglobin oxidation in the RBCs. This restoration of thiol-depleted RBCs by externally applied NAC amide was significantly greater than that found using NAC. Unlike NAC, NAC amide protected hemoglobin from oxidation. (L. Grinberg et al., Free Radic Biol Med., 2005 Jan. 1, 38(1):136-45). In a cell-free system, NAC amide was shown to react with oxidized glutathione (GSSG) to generate reduced glutathione (GSH). NAC amide readily permeates cell membranes, replenishes intracellular GSH, and, by incorporating into the cell's redox machinery, protects the cell from oxidation. Because of its neutral carboxyl group, NAC amide possesses enhanced properties of lipophilicity and cell permeability. (See, e.g., U.S. Pat. No. 5,874,468 to D. Atlas et al.). NAC amide is also superior to NAC and GSH in crossing the cell membrane, as well as the blood-brain barrier.

NAC amide may function directly or indirectly in many important biological phenomena, including the synthesis of proteins and DNA, transport, enzyme activity, metabolism, and protection of cells from free-radical mediated damage. NAC amide is a potent cellular antioxidant responsible for maintaining the proper oxidation state within cells. NAC amide is synthesized by most cells and can recycle oxidized biomolecules back to their active reduced forms. As an antioxidant, NAC amide may be as effective, if not more effective, than GSH.

In one embodiment of the present invention, a method is provided to increase the intracellular concentration of GSH in gametes, particularly oocytes by supplementing the oocyte culture medium with NAC amide. (Example 1). It will be understood that NAC amide can be in a composition, preparation, or formulation that is added to the culture medium. NAC amide, and physiologically acceptable derivatives, salts, or esters thereof; are suitable for use according to the present invention. NAC amide is also water-soluble. That NAC amide can increase the intracellular concentration of glutathione is an advantage of this invention, because an increase in intracellular glutathione concentration can reduce oxidative stress and thus enhance the fertilization process and early embryo development. In accordance with this invention, NAC amide supplementation functions to reduce oxidative stress that leads to decreased oocyte quality, decreased fertilization and decreased embryo viability in in vitro systems.

The term “embryo” refers to the early stages of growth of an organism, including human and non-human mammals, following fertilization up to the blastocyst stage. An embryo is characterized by having totipotent cells, which are undifferentiated. In contrast, somatic cells of an individual are differentiated cells of the body that are not totipotent.

In another embodiment, the present invention encompasses a culture medium composition comprising NAC amide, or a physiologically acceptable salt or ester thereof, for in vitro fertilization, in particular, applied to the culture of oocytes (ova) or early embryos (fertilized oocytes), or to the pretreatment of oocytes or sperm. In particular, the culture medium composition is effective for the stimulation of the growth and qualitative stabilization of early embryos and is suitable for the culture of early embryos in vitro.

In another embodiment, the present invention encompasses a method for improving sperm function, wherein sperm have an increased capability to fertilize an oocyte. This function may be assayed by a broad range of measurable cell functions. Such assayable functions include sperm motility, sperm viability, membrane integrity of sperm, in vitro fertilization, sperm chromatin stability, survival time in culture, penetration of cervical mucus, as well as sperm penetration assays and hemizona assays. Sperm have improved function after exposure to a composition or method if they perform significantly better (p<0.05) with a PCAGH, compared to a control (i.e., assay performed without including a PCAGH). A description of various, representative assays that may be used to assess sperm function are disclosed in U.S. Pat. No. 6,539,309 to J. E. Ellington et al. and are set forth in Example 1 herein.

In those embodiments in which NAC amide is formulated into a lubricant to reduce oxidative stress and free radical formation prior, during, or after fertilization, the base of the lubricant is a nonspermicidal lubricious compound. Such lubricants include petroleum jelly, vegetable oil, glycerin, polycarbophil, hydroxyethyl cellulose, methylcellulose, silicon oil, carbomer (e.g., carbomer 934), alginate, methylparaben, palm oil, cocoa butter, aloe vera, other plant oils, alginate propylene glycol, unibase (Warner-Chilcott), mineral oil, a combination of polyethylene oxide, sodium carboxypolymethylene and methylparaben, and the like. For example, a base lubricant of 50% petroleum jelly/50% glycerin is suitable. Additional ingredients, such as pH stabilizers and anti-oxidants, may be added. Sodium hydroxide is preferably added to bring the pH to 7.4. Other pH stabilizers include EDTA or zwitterionic buffers (e.g., TES, PIPES, MOPS, HEPES). Other anti-oxidants or free-radical scavengers, e.g., vitamin E, may be added. In certain embodiments, silicon oil or polyvinyl alcohol is added.

The lubricant is preferably non-irritating and easily applied. It may be in the form of a gel, foam, cream, jelly, suppository (See, U.S. Pat. No. 4,384,003 to Kazrmiroski), or the like. The lubricant may be packaged in a kit containing a tube of lubricant and an applicator for intra-vaginal application, e.g., for use during coitus or artificial insemination. It may also be used during the collection of sperm from sperm donors by a variety of means. In addition, the lubricant may be used in various assisted reproductive techniques and diagnostic procedures. For example, the lubricant may be used to coat a catheter for insertion into a bladder for retrograde sperm collection. It may be used to lubricate a catheter, pipette or hand, prior to performing embryo transfer, artificial insemination, or diagnostic procedures such as endoscopy, contrast radiography or biopsy. The lubricant may be used in any animal species for sperm collection, coitus, assisted reproductive techniques and the like. Animals include, but are not limited to, humans, bovine, equine, canine, ovine, avian, feline, and various exotic or rare species (e.g., elephant, lion, rhinoceros).

In another embodiment of this invention, methods for extending sperm (e.g., to dilute or suspend the sperm) to obtain sperm with improved function are provided. Improved function of sperm refers to the improved potential of a sperm to fertilize an oocyte. This potential may be assessed by motility, viability, survival time, membrane stabilization, levels of lipid peroxidation damage, chromatin stability, mucus penetration, oocyte fertilization or subsequent embryonic development and the like, as described in Example 2. Similarly, improved function of an oocyte refers to the improved potential for fertilization of the oocyte by sperm, followed by normal development. Improved function of an embryo refers to improved potential for normal development and offspring production. This potential for oocytes and embryos is assessed by evaluating chromosome numbers, cell numbers, cytoskeleton formation and metabolic activity. Improved function can also refer to the enhanced performance, viability and survival of sperm, oocytes or embryos as a result of the presence of NAC amide in the culture medium or lubricant, as assessed by various assays compared with appropriate controls.

Extending sperm is used to resuspend a sperm pellet following isolation or washing, to dilute a semen sample, to dilute a culture of sperm, and the like. In this way, sperm are placed into a medium, or a medium containing NAC amide, suitable for a variety of procedures, including culture, insemination, assays of fertilization potential as described herein, in vitro fertilization, freezing, intrauterine insemination, cervical cap insemination, and the like. The sperm may be added to the medium or the medium may be added to the sperm.

In other embodiments, the present invention encompasses methods for the culture of extended sperm to increase their survival during holding or culture at a range of temperatures from about room temperature (e.g., 20° C.) to about body temperature (e.g., 37° C. or 39° C.). This includes culture of sperm in toxicity screen tests and the holding of sperm for sorting into X and Y chromosome-containing fractions by flow cytometry for generating sexed offspring. Further, sperm extending medium is used for preparing sperm for direct insemination, cryopreservation, and for intracytoplasmic sperm injection (ICSI) which requires a more viscous medium to slow motile sperm down for pick-up by the transfer pipette for injection into the egg. Sample media include, but are not limited to, balanced salt solution which may contain zwitterionic buffers, such as TES, HEPES, PIPES; other buffers, such as sodium bicarbonate; TALP; or HTF. Additional ingredients may include macromolecules, for example, albumin, oviductin, gelatin, hyaluronic acid, milk, egg yolk, hormones, additional free radical scavengers (e.g., melanin, vitamin E derivatives, thioredoxine), enzymes (e.g., SOD, catalase), growth factors (e.g., EGF, IGF, PAF, VIP), polymeric molecules (e.g., heparin, dextran, polylysine, PVP or PVA). Additionally, such media may include sperm motility stimulants such as caffeine, follicular fluid, calcium, oxytocin, kallikrinen, prostaglandins, thymus extracts, pentoxyfilline, 2-deoxyadenosine, inositol, flavanoids, platelet activating factor, hypotaurine, chondroitin sulfate, and mercaptoethanol. Caffeine (e.g., 5 mM) and pentoxyfilline (e.g., 1 mM) are suitable stimulants. Antibiotics and antimycotics may also be included.

In another embodiment, this invention embraces methods for increasing the survival and maturation of oocytes, embryos or embryonic stem cells (ESC) in in vitro culture systems. Oocytes, embryos, or ESC are cultured for use in various diagnostic and toxicology assays, in vitro fertilization, or for the propagation of offspring. These methods comprise contacting a sample containing an oocyte, an embryo or ESC with a culture medium that includes NAC amide or a physiologically acceptable derivative or salt or ester thereof.

In accordance with the methods and compositions of the present invention, NAC amide is administered, supplied, or used in conjunction with another component or factor, e.g., granulocyte-macrophage-colony stimulating factor (GM-CSF), for increasing the viability and success of embryo development to the blastocyst stage and beyond.

In another embodiment, NAC amide is used in methods and compositions involved in the production and maintenance of transgenic animal embryos and eggs, including non-human transgenic animals such as pigs, sheep, goats and rodents as nonlimiting examples. The eggs and embryos of transgenic animals typically have low levels of naturally produced GSH and low success rates for full development. Thus, in accordance with this embodiment, NAC amide supplied to the eggs and embryos of transgenic animals will improve the rate of full development of transgenic organisms during in vitro culture, as well as in in vivo, thereby increasing the rate of success in achieving full term transgenic animals. The present invention also allows for the production of transgenic animals having the ability to produce, for example, human and animal amino acids, heterologous proteins, e.g., clotting factors, growth factors, anti-cancer factors, etc. Transgenic animals produced in accordance with this invention can also be used as a source of antigen-free organs for human transplants.

In another embodiment, the present invention encompasses methods and compositions comprising NAC amide to nurture and support stem cell or other germ cell transplantation into animals, including humans, as well as to support cell growth and cloning in vitro and in vivo.

In another embodiment, the present invention encompasses a pharmaceutically acceptable composition comprising NAC amide, or a physiologically acceptable derivative or salt or ester thereof, used in procedures to prevent, reduce, counteract, or alleviate oxidative stress resulting from excesses of heme oxygenase and bilirubin, which adversely affect the survival and development of preterm neonates. Administration of NAC amide to neonates can further serve to improve bronchopulmonary dysplasia in preterm infants and neonates by improving and supplementing their antioxidant defense and by preventing increased susceptibility to infection and inflammation. NAC amide provided to such newborns and preterm infants can also prevent apoptosis and its debilitating and tragic effects.

In accordance with the invention, for treatment purposes, NAC amide may be administered by several routes that are suited to the treatment or therapy method, as will be appreciated by the skilled practitioner. Nonlimiting examples of routes and modes of administration for NAC amide include parenteral routes of injection, including subcutaneous, intravenous, intramuscular, and intrasternal. Other modes of administration include, but are not limited to, oral, inhalation, topical, intranasal, intrathecal, intracutaneous, opthalmic, vaginal, rectal, percutaneous, enteral, injection cannula, continuous infusion, timed release and sublingual routes. In one embodiment of the present invention, administration of NAC amide may be mediated by endoscopic surgery. For the treatment of various neurological diseases or disorders that affect the brain, NAC amide can be introduced into the tissues lining the ventricles of the brain. The ventricular system of nearly all brain regions permits easier access to different areas of the brain that are affected by the disease or disorder. For example, for treatment, a device, such as a cannula and osmotic pump, can be implanted so as to administer a therapeutic compound, such as NAC amide, as a component of a pharmaceutically acceptable composition. Direct injection of NAC amide is also encompassed. For example, the close proximity of the ventricles to many brain regions is conducive to the diffusion of a secreted or introduced neurological substance in and around the site of treatment by NAC amide.

For administration to a recipient, for example, injectable administration, a composition or preparation formulated to contain water-soluble NAC amide is typically in a sterile solution or suspension. Alternatively, NAC amide can be resuspended in pharmaceutically- and physiologically-acceptable aqueous or oleaginous vehicles, which may contain preservatives, stabilizers, and material for rendering the solution or suspension isotonic with body fluids (i.e. blood) of the recipient. Non-limiting examples of excipients suitable for use include water, phosphate buffered saline (pH 7.4), 0.15M aqueous sodium chloride solution, dextrose, glycerol, dilute ethanol, and the like, and mixtures thereof. Illustrative stabilizers are polyethylene glycol, proteins, saccharides, amino acids, inorganic acids, and organic acids, which may be used either on their own or as admixtures.

Formulations comprising NAC amide for topical administration may include but are not limited to lotions, ointments, gels, creams, suppositories, drops, liquids, sprays and powders. NAC amide may be administered to mucous membranes in the form of a liquid, gel, cream, and jelly, absorbed into a pad or sponge. Conventional pharmaceutical carriers, aqueous, powder or oily bases, thickeners and the like may be necessary or desirable. Compositions comprising NAC amide for oral administration include powders or granules, suspensions or solutions in water or non-aqueous media, sachets, capsules or tablets. Thickeners, diluents, flavorings, dispersing aids, emulsifiers or binders may be desirable. Formulations for parenteral administration may include, but are not limited to, sterile solutions, which may also contain buffers, diluents and other suitable additives.

Doses, amounts or quantities of NAC amide, as well as the routes of administration used, are determined on an individual basis, and correspond to the amounts used in similar types of applications or indications known to those having skill in the art. As is appreciated by the skilled practitioner in the art, dosing is dependent on the severity and responsiveness of the condition to be treated, but will normally be one or more doses per day, with course of treatment lasting from several days to several months, or until a cure is effected or a diminution of disease state is achieved. Persons ordinarily skilled in the art can easily determine optimum dosages, dosing methodologies and repetition rates. For example, a pharmaceutical formulation for orally administrable dosage form can comprise NAC amide, or a pharmaceutically acceptable salt, ester, or derivative thereof in an amount equivalent to at least 25-500 mg per dose, or in an amount equivalent to at least 50-350 mg per dose, or in an amount equivalent to at least 50-150 mg per dose, or in an amount equivalent to at least 25-250 mg per dose, or in an amount equivalent to at least 50 mg per dose. NAC amide can be administered to both human and non-human mammals. It therefore has application in both human and veterinary medicine.

Examples of suitable esters of NAC amide include alkyl and aryl esters, selected from the group consisting of methyl ester, ethyl ester, hydroxyethyl ester, t-butyl ester, cholesteryl ester, isopropyl ester and glyceryl ester.

In general, a suitable medium for extending sperm or culturing sperm, oocytes, embryos or ESC is a balanced salt solution, such as M199, Synthetic Oviduct Fluid, PBS, BO, Test-yolk, Tyrode's, HBSS, Ham's F10, HTF, Menezo's B2, Menezo's B3, Ham's F12, DMEM, TALP, Earle's Buffered Salts, CZB, KSOM, BWW Medium, and emCare Media (PETS, Canton, Tex.). In one embodiment, M199 medium is used for culturing oocytes. In certain embodiments, TALP or HTF is used for sperm culture medium, and CZB is used for embryo culture medium.

The concentration of the NAC amide in the culture medium for oocytes or embryos ranges from 0.001-15%, or 0.001-10%, or 0.001-5%, or 0.01-5%, or 0.05-1%, or 0.05-0.5%, or 0.1-5%, or 0.1-1%, as appropriate. Optionally, other additives may be present such as amino acids (e.g., glutamic acid). Generally, the additives include, without limitation, macromolecules, buffers, antibiotic and possibly a sperm stimulant if fertilization is to be achieved. Hormones or other proteins may also be added. Such hormones and proteins include luteinizing hormone, estrogen, progesterone, follicle stimulating hormone, human chorionic gonadotropin, growth factors, follicular fluid and oviductin, albumin and amino acids. Generally, the medium also contains serum from about 1% to 20%. Preferably, the serum is from the same animal source as is the oocyte or embryo source. Sperm, oocytes, or embryos are typically cultured in such media in 5% CO₂ and humidified air at 37° C. Cultures may further contain a feeder layer comprising somatic cells, generally irradiated cells, cultured cells, or cells with a limited life span in culture (e.g., thymocytes).

In other embodiments, this invention encompasses methods for reducing losses of functional sperm, reducing cellular damage to an oocyte, or reducing cellular damage to an embryo or ESC (embryo stem cell) resulting from storage in a refrigerated, frozen or vitrified state. The methods comprise combining a PCAGH in an amount effective to reduce loss or damage with a sample containing sperm, oocyte, embryo or ESC, and storing the sample in a refrigerated, frozen or vitrified state.

NAC amide may be an additive in cyropreservation media for sperm, oocytes, embryos, and ESC. Cryoprotective medium is typically added slowly to the cells in a drop wise fashion. Such cryoprotective media comprise permeating and nonpermeating compounds. Most commonly, DMSO, glycerol, propylene glycol, ethylene glycol, or the like are used. Other permeating agents include propanediol, dimethylformamide and acetamide. Nonpermeating agents include polyvinyl alcohol, polyvinyl pyrrolidine, anti-freeze fish or plant proteins, carboxymethylcellulose, serum albumin, hydroxyethyl starch, Ficoll, dextran, gelatin, albumin, egg yolk, milk products, lipid vesicles, or lecithin. Adjunct compounds that may be added include sugar alcohols, simple sugars (e.g., sucrose, raffinose, trehalose, galactose, and lactose), glycosaminoglycans (e.g., heparin, chrondroitin sulfate), butylated hydroxy toluene, detergents, free-radical scavengers, additional anti-oxidants (e.g., vitamin E, taurine), amino acids (e.g., glycine, glutamic acid), and flavanoids and taxol (preferably 0.5-5 μm). Glycerol is preferred for sperm freezing, and ethylene glycol or DMSO for the freezing of oocytes, embryos, or ESC. Typically, glycerol is added at 3-15%; other suitable concentrations may be readily determined using known methods and assays. Other agents are added typically at a concentration range of approximately 0.1-5%. Proteins, such as human serum albumin, bovine serum albumin, fetal bovine serum, egg yolk, skim milk, gelatin, casein or oviductin, may also be added

Following suspension of the cells in the cryoprotective medium (e.g., for storage), the container is sealed and subsequently either refrigerated or frozen. Briefly, for refrigeration, the sample is placed in a refrigerator in a container filled with water for one hour or until the temperature reaches 4° C. Samples are then placed in Styrofoam containers with cool packs and may be shipped for insemination, in the case of sperm, the next day. If the sample is to be frozen, the cold sample is aliquoted into cryovials or straws and placed in the vapor phase of liquid nitrogen for one to two hours, and then plunged into the liquid phase of liquid nitrogen for long-term storage or frozen in a programmable computerized freezer. Frozen samples are thawed by warming in a 37° C. water bath and are directly inseminated or washed prior to insemination. Other cooling and freezing protocols may be used. Vitrification involves dehydration of the oocyte or embryos using sugars, Ficoll, or the like. The oocyte or embryo is then added to a cryoprotectant and rapidly moved into liquid nitrogen.

In accordance with the methods and compositions of present invention, sperm, oocytes, or embryos may be prepared and stored as described above. Refrigeration is generally an appropriate means for short-term storage, while freezing or vitrification are generally appropriate means for long or short-term storage.

The compositions and methods of the present invention increase fertility of animals. These methods are generally applicable to many species, including human, bovine, canine, equine, porcine, ovine, avian, rodent and others. Although useful whenever fertilization is desired, the present invention has particular use in animals and humans that have a fertilization dysfunction in order to increase the likelihood of conception. Such dysfunctions include low sperm count, reduced motility of sperm, and abnormal morphology of sperm. In addition to these dysfunctions, the methods and compositions of the present invention are useful in artificial insemination procedures. Often, in commercial breedings, the male and female are geographically distant requiring the shipment of sperm for insemination. Because of the extended period of time between obtaining a sperm sample and insemination, shipment in refrigerated or frozen state is necessary. Moreover, for particularly valuable or rare animals, long-term storage may be desirable. For humans, geographical distance or time considerations may necessitate storage of sperm. Men with diseases where radiation treatment is part of therapy or prior to vasectomies may desire to have sperm stored for future use. After frozen storage, gamete cells are often cultured during end use. Survival and health of the gamete cells in culture can be improved by addition of NAC amide to the culture and/or cryopreservative medium.

The lubricant according to the present invention is useful for all situations involving sperm collection, coitus, and artificial insemination. Currently, sperm collection is done without any lubrication because of the spermicidal nature of commercial lubricants and saliva (Goldenberg et al., Fertility and Sterility 26:872-723, 1975, Scoeman & Tyler, J. Reprod. Fert. 2:275-281, 1985, Miller et al., Fert. and Steril. 61:1171-1173, 1994). The use of a non-spermicidal lubricant containing NAC amide so as to improve sperm function and increase potential fertility is desirable for the comfort of the donor. As such, the lubricant may be applied to condoms or other collection devices, such as catheters or vials. Infertile couples also often have the need for lubricants. However, because lubricants are spermicidal, they are not recommended for use. In these cases, the application of a lubricant intravaginally, with or without an applicator, would be desirable and beneficial because sperm function would be increased. Similarly, the lubricant may be applied intravaginally prior to artificial insemination to improve the chances of conception.

Supplementation of culture, fertilization and maturation media with NAC amide provides an environment for oocytes, sperm and embryos that allows their prolonged viability, survivability, normalcy and function during the time that they are in culture before, during and after in vitro fertilization and embryo development, and prior to transfer into the female. That NAC amide is superior to other antioxidants, such as GSH and NAC, is supported by Example 1 herein. The present invention encompassing the use of NAC amide as a supplement in maturation medium for embryos permits the embryos to continue development and have improved function until the blastocyst stage, compared with control, unsupplemented medium. (Example 1).

The following examples further describe the invention and are not intended to limit the invention in any way.

### EXAMPLES

### Example 1

This Example describes an evaluation of the effects of NAC amide, glutathione (GSH) and N-acetylcysteine (NAC) supplementation to incubation and culture media during porcine oocyte maturation, fertilization and embryo culture on various measures of fertilization and embryo development, as well as on the intracellular concentration of GSH.

Experimental Design: Three trials were conducted, each trial utilizing 30 porcine oocytes per treatment group (90 total oocytes per each of four treatment groups). Oocytes were purchased from Trans Ova Genetics, Sioux City, Iowa. Treatment groups were: 1) Control (no supplemental anti-oxidants); 2) GSH supplementation (1.0 mM); 3) NAC supplementation (1.0 mM); and 4) NAC amide supplementation (1.0 mM).

Chemicals: All chemicals, unless otherwise specified, were obtained from Sigma Chemical Company (St. Louis, Mo.) and were of embryo grade quality. NAC amide was supplied by Dr. Glenn Goldstein. NAC amide can be prepared, for example, as described in U.S. Pat. No. 6,420,429 to D. Atlas et al., the contents of which are incorporated herein by reference.

In vitro Maturation: Oocytes were maturated for 20 to 24 hours in tissue culture medium 199 with Earle's salts, 0.01 U/mL LH and FSH, 10 ng/mL EGF, antibiotics, and 10% fetal calf serum under mineral oil (Specialty Media, Phillipsburg, N.J.) at 39° C. in an atmosphere of 5% CO₂ and then for an additional 20 to 24 h without hormones.

In vitro Fertilization (IVF) of Oocytes: Cumulus cells by were removed by agitation with 0.1% hyaluronidase, washed and placed in Tris-fertilization medium (113.1 mM NaCl, 3 mM KCl, 7.5 mM CaCl₂.2H₂O, 20 mM Tris, 11 mM D (+)-glucose, 5 mM sodium pyruvate, 1 mg/mL BSA, 2 mM caffeine) with mineral oil overlay and freeze-thawed spermatozoa were added at a concentration of 2000 spermatozoa/oocyte. The gametes were incubated at 39° C. in an atmosphere of 5% CO₂ for approximately 6 hours.

In vitro Fertilization Parameter Evaluation: Fertilization was analyzed 12 hours after IVF by fixing the oocytes on a microscope slide with 25% (v:v) acetic acid in ethanol at room temperature for 48 hours. Oocytes were stained with 1% orcein in 45% (v:v) acetic acid and examined using a phase-contrast microscope at 400× magnification.

In vitro Culture: Putative zygotes were washed and incubated in NCSU-23 culture medium (108.73 mM NaCl, 4.78 mM KCl, 1.19 mM KH₂PO₄, 1.19 mM MgSO₄.7H₂O, 5.5 mM glucose, 1 mM glutamine, 7 mM taurine 5 mM hypotaurine, 25.07 mM NaHCO₃, 1.7 mM CaCl₂.2H₂O, 75 μg/mL Penicillin G, 50 μg/mL Streptomycin, 4 mg/mL BSA, pH 7.4) with mineral oil overlay at 39° C. in an atmosphere of 5% CO₂ for 48 hours. After 48 hours, embryos that had undergone the first cell division were placed in fresh NCSU 23 culture media in the same manner as described above until blastocyst formation, 144 hours post-IVF.

Glutathione Assay: Oocytes were washed in PBS, frozen, and ruptured using a blunt glass rod in phosphoric acid. The assay was performed as described previously (B. D. Whitaker and J. W. Knight, 2004, Theriogenology, 62:311-322) and the amount of GSH was determined using a standard curve of concentration GSH versus rate of change in absorbency.

Table 1 summarizes the intracellular GSH concentration per oocyte expressed as pmol. Supplementation with NAC amide resulted in a 2.2-fold increase in intracellular GSH concentration compared with control. Of the supplements examined, NAC amide resulted in a 40% greater increase in intracellular GSH compared with supplementation of GSH per se and a 15% greater intracellular GSH concentration compared with NAC supplementation.

TABLE 1

| | GSH concentration/ |
|---|---|
| Treatment | oocyte (pmol) |
| Control | 3.19 |
| GSH | 4.18 |
| NAC | 6.00 |
| NAC amide | 7.03 |

NAC amide and NAC are significantly (P<0.05) higher than control. NAC amide is significantly (P<0.05) higher than GSH.

Fertilization parameters were subjectively examined by nuclear staining samples (n=4) of putative zygotes from each of the treatment groups 12 hours after IVF was complete (Illustration 1, * indicates pronucleus). In the preliminary analysis, only a small number of zygotes were subjected to staining, since the intent of the studies described herein was to assess the number of zygotes that continued development to the 2-cell and blastocyst stages. This analysis was simply to see if any obvious anomalies were occurring. Supplementation of medium with GSH, NAC, or NAC amide did not have any noticeable changes on fertilization events based upon the small number of zygotes that were preliminarily subjected to nuclear staining.

Based on previous findings (B. D. Whitaker and J. W. Knight, 2004, Theriogenology, 62:311-322) that increasing glutathione concentrations in the oocyte decreases the incidence of polyspermy, along with literature reports that glutathione promotes the oocyte-sperm complex to develop the male pronucleus after IVF, it is encouraging that NAC amide may play a beneficial role in these processes.

The remaining zygotes were cultured through the blastocyst stage of development (148 hours) in their respective media and their development and viability progress was recorded (Table 2). NAC amide supplementation of the culture medium enhanced the development of zygotes to the 2-cell stage and further aided the subsequent final percentage of those embryos reaching the 2-cell stage that continued development onto the blastocyst stage (the endpoint of in vitro analysis).

TABLE 2

| | | % embryos reaching |
|---|---|---|
| | | blastocyst stage of |
| | % embryos reaching | development (of |
| | 2-cell stage | those in observed in |
| Treatment | of development | the 2-cell stage) |
| Control | 19 | 40 |
| GSH | 25 | 40 |
| NAC | 30 | 55 |
| AD4 | 45 | 85 |

NAC amide resulted in a significantly greater % of embryos developing to the 2-cell (P<0.05) and blastocyst (P<0.10) stages.

The results from the studies in Example 1 show that supplementation of culture medium with NAC amide significantly increased the intracellular concentration of glutathione. This is a biologically important finding since there is ample evidence to indicate that increasing intracellular glutathione concentrations will reduce oxidative stress and hence enhance the fertilization process and early embryonic development. The findings presented in this Example demonstrate that media supplementation with NAC amide increased the percentage of zygotes that cleaved to become 2-cell embryos. Most importantly, 85% of those embryos cultured in medium supplemented with NAC amide continued development to the endpoint of reaching the blastocyst stage of development. This was more than twice the percentage of control (unsupplemented) embryos that developed to the blastocyst stage.

Among the three antioxidants examined (GSH, NAC and NAC amide), NAC amide was consistently more effective than the two naturally occurring products. These results are similar to results of other studies in which GSH per se (versus other γ-glutamyl cycle compounds) was only marginally effective (compared with unsupplemented control medium). (B. D. Whitaker and J. W. Knight, 2004, Theriogenology, 62:311-322). Although NAC supplementation did enhance all parameters measured, it did so to a lesser degree than did NAC amide.

These results to date strongly suggest that by increasing intracellular concentration of glutathione in the oocyte, NAC amide reduces the oxidative stress that leads to decreased oocyte quality, fertilization, and embryo viability in in vitro systems.

### Example 2

This Example describes various assays and methods that are used to assess sperm function/fertilization potential. Further description may be found in U.S. Pat. No. 6,593,309 to J. E. Ellington et al.). Sperm motility is one function that may be used to assess sperm function and thus fertilization potential. Motility of sperm is expressed as the total percent of motile sperm, the total percent of progressively motile sperm (swimming forward), or the speed of sperm that are progressively motile. These measurements may be made by a variety of assays, but are conveniently assayed in one of two ways. Either a subjective visual determination is made using a phase contrast microscope when the sperm are placed in a hemocytometer or on a microscope slide, or a computer assisted semen analyzer is used. Under phase contrast microscopy, motile and total sperm counts are made and speed is assessed as fast, medium or slow. Using a computer assisted semen analyzer (Hamilton Thorn, Beverly, Mass.), the motility characteristics of individual sperm cells in a sample are objectively determined. The analyzer tracks individual sperm cells and determines motility and velocity of the sperm. Data are expressed as percent motile, and measurements are obtained for path velocity and track speed as well.

Sperm viability is measured in one of several different methods. By way of example, two of these methods are staining with membrane exclusion stains and measurement of ATP levels. Briefly, a sample of sperm is incubated with a viable dye, such as Hoechst 33258 or eosin-nigrosin stain. Cells are placed in a hemocytometer and examined microscopically. Dead sperm with disrupted membranes stain with these dyes. The number of cells that are unstained is divided by the total number of cells counted to give the percent live cells. ATP levels in a sperm sample are measured by lysing the sperm and incubating the lysate with the luciferase enzyme, which fluoresces in the presence of ATP. The fluorescence is measured in a luminometer (Sperm Viability Test; Firezyme, Nova Scotia, Canada). The amount of fluorescence in the sample is compared to the amount of fluorescence in a standard curve allowing a determination of the number of live sperm present in the sample.

Membrane integrity of sperm is typically assayed by a hypo-osmotic swell test that measures the ability of sperm to pump water or salts if exposed to non-isotonic environments. Briefly, in the hypo-osmotic swell test, sperm are suspended in a solution of 75 mM fructose and 25 mM sodium citrate, which is a hypo-osmotic (150 mOsm) solution. Sperm with intact, healthy membranes pump salt out of the cell causing the membranes to shrink as the cell grows smaller. The sperm tail curls inside this tighter membrane. Thus, sperm with curled tail are counted as live, healthy sperm with normal membranes. When compared to the total number of sperm present, a percent of functional sperm may be established.

The degree of membrane integrity is preferably determined by lipid peroxidation (LPO) measurements that assess sperm membrane damage generated by free radicals released during handling. Lipid membrane peroxidation is assayed by incubating sperm with ferrous sulfate and ascorbic acid for one hour in a 37° C. water bath. Proteins are precipitated with ice-cold trichloroacetic acid. The supernatant is collected by centrifugation and reacted by boiling with thiobarbituric acid and NaOH. The resultant malondialdehyde (MDA) formation is quantified by measuring absorbance at 534 nm, compared to an MDA standard (M. Bell et al., J. Andrology 14:472-478, 1993). LPO is expressed as nM MDA/108 sperm. A stabilizing effect of PCAGHs results in decreased LPO production. According to the present invention, when used in a medium or environment in which sperm are placed, NAC amide can reduce or alleviate the oxidative stress (peroxidation) that is encountered by sperm during handling

The stability of chromatin DNA is assayed using the sperm chromatin sensitivity assay (SCSA). This assay is based on the metachromatic staining of single and double stranded DNA by acridine orange stain, following excitation with 488 nm light. Green fluorescence indicates double stranded DNA, and red fluorescence indicates single stranded DNA. The extent of DNA denaturation in a sample is expressed as “α” and calculated by the formula α=red/(red+green). In all cases, sperm are mixed with TNE buffer (0.01 M Tris aminomethane-HCl, 0.015M NaCl, and 1 mM EDTA) and flash frozen. Sperm samples are then subjected to 0.01% Triton-X, 0.08N HCl and 0.15M NaCl, which induces partial denaturation of DNA in sperm with abnormal chromatin. Sperm are stained with 6 g/ml acridine orange and run through a flow cytometer to determine “α”.

In vitro fertilization rates are determined by measuring the percent fertilization of oocytes in vitro. Maturing oocytes are cultured in vitro in M199 medium plus 7.5% fetal calf serum and 50 μg/ml luteinizing hormone for 22 hours. Following culture for 4 hours, the sperm are chemically capacitated by adding 10 IU of heparin and incubated with oocytes for 24 hours. At the end of the incubation, oocytes are stained with an aceto-orcein stain, or equivalent, to determine the percent oocytes fertilized. Alternatively, fertilized oocytes may be left in culture for 2 days, during which time division occurs and the number of cleaving embryos (i.e., 2 or more cells) are counted.

Survival time in culture of sperm (time to loss of motility) is another convenient method of establishing sperm function. This parameter correlates well with actual fertility of a given male. Briefly, an aliquot of sperm is placed in culture medium, such as Tyrode's medium, pH 7.4 and incubated at 37° C., 5% CO₂, in a humidified atmosphere. At timed intervals, for example every 8 hours, the percentage of motile sperm in the culture is determined by visual analysis using an inverted microscope, or with a computer assisted sperm analyzer. As an endpoint, a sperm sample is considered no longer viable when less than 5% of the cells have progressive motility.

Another parameter of sperm function is the ability to penetrate cervical mucus. This penetration test can be done either in vitro or in vivo. Briefly, in vitro, a commercial kit containing cervical mucus (Tru-Trax, Fertility Technologies, Natick, Mass.), typically bovine cervical mucus, is prepared. Sperm are placed at one end of the track and the distance that sperm have penetrated into the mucus after a given time period is determined. Alternatively, sperm penetration of mucus may be measured in vivo in women. At various times post-coitus, a sample of cervical mucus is removed and examined microscopically for the number of sperm present in the sample. In the post-coital test, improved sperm function is established if more sperm with faster velocity are seen in the mucus sample after exposure to a PCAGH lubricant versus a sample of mucus from the patient after exposure to a control lubricant.

Other assays of sperm function potential include the sperm penetration and hemizona assays. In the sperm penetration assay, the ability of sperm to penetrate into an oocyte is measured. Briefly, commercially available zona free hamster oocytes are used (Fertility Technologies, Natick, Mass.). Hamster oocytes are suitable in this assay for sperm of any species. Capacitated sperm, such as those cultured with bovine serum albumin for 18 hours, are incubated for 3 hours with the hamster oocytes. Following incubation, oocytes are stained with acetolacmoid or equivalent stain and the number of sperm penetrating each oocyte is counted microscopically. A hemizona assay measures the ability of sperm to undergo capacitation and bind to an oocyte. Briefly, in this assay, live normal sperm are incubated in media with bovine serum albumin, which triggers capacitation. Sperm are then incubated with dead oocytes that are surrounded by the zona pellucida, an acellular coating of oocytes. Capacitated sperm bind to the zona and the number of sperm binding is counted microscopically.

### Example 3

This Example describes methods for washing and isolating sperm and sperm-containing samples to obtain sperm-rich samples and samples of the most motile sperm. Such samples contain sperm with improved function. Sperm are washed by contacting a sample containing sperm with a polysaccharide-containing solution, wherein the polysaccharide is not arabinogalactan. (U.S. Pat. No. 6,593,309 to J. E. Ellington et al.). Motile sperm are isolated by contacting a sample containing sperm with a media solution comprising a polysaccharide, wherein the polysaccharide is not arabinogalactan, and subjecting the mixture to conditions sufficient to separate the sperm. Such media include, but are not limited to, Tyrode's albumin lactate phosphate (TALP), human tubal fluid (HTF; Fertility Technology, Natick, Mass.), Ham's F10, Ham's F12, Earle's buffered salts, Biggers, Whitten and Whitingham (BWW), CZB, T6, Earle's MTF, KSOM, SOF, and Benezo's B2 or B3 media. Formulas for these media are well known, and preformulated media may be obtained commercially (e.g., Gibco Co. or Fertility Technologies, Natick, Mass.). In addition, a zwitterionic buffer (e.g., MOPS, PIPES, HEPES) may be added. The polysaccharides may include pectin, gum guar, or gum arabic for isolating and washing sperm. Gum arabic may be added to about 20%, or gum guar is added to about 5%. NAC amide can be added as the antioxidant component.

These media may further contain a macromolecule as long as the solution remains a balanced salt solution. Such macromolecules include polyvinyl alcohol, albumin (bovine serum albumin or human serum albumin), oviductin (Gandolfi et al., Repro. Fert. Dev. 5:433, 1993), superoxide dismutase, vitamin E, gelatin, hyaluronic acid, catalase, egg yolk, casein, or other protein. Albumin or gelatin is added generally at 0.5% and hyaluronic acid or polyvinylalcohol at 1.0%; the other macromolecules are added at similar concentrations (e.g., 0.05-5%). Sperm isolation media contain at least one polysaccharide at about 0.01-5% (e.g., 0.1-5%, 0.1-1%, 1%-5%) in addition to either a density gradient compound for centrifugation methods, or a macromolecule for swim-up isolation methods. Density gradient materials are generally added to a concentration of 5-90%. Such materials include dextran, iodixanol, sucrose polymers, nycodenz, or polyvinylpyrrolidone coated silica (i.e., Percoll). In typical applications, a sperm containing solution is layered over a gradient material, preferably Percoll at 30-90%, mixed with 0.05% pectin, and then subjected to centrifugation to collect sperm with improved function. When sperm swim-up is used to isolate sperm, a macromolecule, such as those discussed above, is added. Preferably 1-10 mg/ml of hyaluronic acid is used. Media used in any of these procedures may further comprise a balanced salt solution.

Sperm are washed or isolated by subjecting a sperm containing-medium mixture to conditions sufficient to separate the desired sperm from the sample Briefly, cells are contacted with the solution by placing cells in the solution from a brief time up to incubation for 4 hours. Preferably the temperature at which contacting occurs is from about 20° C. to about 39° C. Following this initial contact, different methods may be used to isolate sperm, such as centrifugation, swim-up, separation columns, and the like. For example, one such method is centrifugation of a sperm sample through a continuous gradient of the solution comprising a polysaccharide, particularly a PCAGH as described in U.S. Pat. No. 6,593,309 to J. E. Ellington et al. In this method, the solution comprising a PCAGH is placed in a centrifuge tube and a semen sample or sperm cells are layered over the medium at approximately a ratio of one part semen (or sample) to one part medium. The tube is centrifuged at approximately 300×g for ten to twenty minutes. A sperm-rich fraction with improved function, and thereby increased fertilization potential, is recovered in a pellet at the bottom of the tube. Because the PCAGH is non-toxic to sperm, a follow-up wash step to remove the PCAGH is not required. Isolation may be performed in a method similar to the above wash process; however, the PCAGH solution can either be layered under the sperm sample, but on top of a density gradient like Percoll, or mixed directly into the Percoll gradient. Alternatively, sperm are isolated by a swim-up method. Briefly, sperm swim-up tubes are prepared by placing 1.5 ml of wash media in a 12×75 mm round bottom tube. Sperm are layered under this wash media using a 27 gauge needle and 1 ml syringe at 1 part sperm suspension to 2 parts wash medium. The tubes are incubated undisturbed for 1 hour. After incubation, the wash medium (that the motile sperm have swum up into) is removed and centrifuged for 10 minutes at 300×g. A final pellet of motile sperm is then recovered for analysis or use. Other methods, such as column separation, may alternatively be used. Sperm may be further washed after isolation of sperm, such as by centrifugation through a Percoll gradient. Washing sperm can be used to transfer sperm from one solution to another. For any of these methods, the sample may be semen, partially purified sperm, or purified sperm. Moreover, sperm suitable in the present invention may be procured from animal species including human, bovine, canine, equine, porcine, ovine, rodent, avian or exotic animals, such as lions, tigers, giraffes, monkeys, zebras, pandas, jaguars, elephants, rhinoceros, and others.

### Example 4

This Example investigates the effects of NAC amide supplementation to culture media during porcine oocyte maturation, fertilization, and embryo culture on intracellular concentrations of GSH after oocyte maturation, IVF parameters, success of intracytoplasmic sperm injection (ICSI), and embryo development following ICSI and pronuclear microinjection.

All chemicals, unless otherwise specified, were obtained from Sigma Chemical Company (St. Louis, Mo.) and were of embryo grade quality. Dr. Glenn Goldstein and associates provided the NAC amide. Oocytes (BoMed, Madison, Wis.) were maturated for 20 to 24 h in tissue culture medium 199 supplemented with Earle's salts, 0.01 U/mL LH and FSH, antibiotics, and 10% fetal calf serum under mineral oil (Specialty Media, Phillipsburg, N.J.) at 39° C. in an atmosphere of 5% CO₂ and then for an additional 20 to 24 h without hormones.

After in vitro maturation, cumulus cells were removed from the oocytes by repeat pipetting in maturation medium containing 0.1% hyaluronidase. Oocytes were then washed in 100 μL drops of 0.2 M sodium phosphate buffer containing 10 mM EDTA (pH 7.2). Approximately 30 oocytes were transferred with 5 tit 0.2 M sodium phosphate buffer containing 10 mM EDTA (pH 7.2) to a 1.5 mL microcentrifuge tube (Fischer Scientific, Pittsburgh, Pa.) and stored at −80° C. until the assay is performed. Each tube contained 5 μL of 1.25 M phosphoric acid and the oocytes were ruptured using a blunt glass rod. The contents of each tube was added to an individual well spectrophotometer tube. The assay was performed as described previously in B. D. Whitaker and J. W. Knight, 2004, Theriogenology, 62:311-322. The absorbency of the samples was read continuously using a spectrophotometer at 412 nm for a total of 10 min. The amount of GSH was then be determined using a standard curve of concentration GSH versus rate of change in absorbency.

Cumulus cells were removed by agitation with 0.1% hyaluronidase, washed and placed in Tris-fertilization medium (113.1 mM NaCl, 3 mM KCl, 7.5 mM CaCl₂.2H₂O, 20 mM Tris, 11 mM D(+)-glucose, 5 mM sodium pyruvate, 1 mg/mL BSA, 2 mM caffeine) with mineral oil overlay and frozen-thawed spermatozoa were added at a concentration of 2000 spermatozoa/oocyte. The gametes were incubated at 39° C. in an atmosphere of 5% CO₂ for approximately 6 h.

Fertilization was analyzed 12 h after IVF by fixing the oocytes on a microscope slide with 25% (v:v) acetic acid in ethanol at room temperature for 48 h. Oocytes were stained with 1% orcein in 45% (v:v) acetic acid and examined using a phase-contrast microscope at 400× magnification.

Cumulus cells were removed by agitation with 0.1% hyaluronidase, washed and placed in microdrops of NCSU-23 culture medium (108.73 mM NaCl, 4.78 mM KCl, 1.19 mM KH₂PO₄, 1.19 mM MgSO₄.7H₂O, 5.5 mM glucose, 1 mM glutamine, 7 mM taurine 5 mM hypotaurine, 25.07 mM NaHCO₃, 1.7 mM CaCl₂.2H₂O, 75 μg/mL Penicillin G, 50 μg/mL Streptomycin, 4 mg/mL BSA, pH 7.4) with mineral oil overlay at 39° C. after centrifugation at 15000×g. Frozen-thawed sperm were then placed in an adjacent microdrop. Manipulation was carried out in 10 μL droplets of HbT under paraffin oil using Narishige manipulators and a Nikon inverted microscope equipped with Hoffman modulator optics. The oocytes were stabilized with a holding pipette with an outer diameter of about 200 μm and an inner diameter of about 50 μm. The sperm were injected using a PiezoDrill micropipette with an outer diameter of 8 to 9 μm and an inner diameter of 6 μm (Humagen, Charlottesville Va.). The polar body of the oocyte was placed at 6 or 12 o'clock and the point of injection was at 3 o'clock. Individual oocytes were penetrated by the injecting micropipette and a small amount of cytoplasm was drawn into the micropipette to ensure penetration of the oocyte. Then, the cytoplasm, together with one sperm and a small amount of medium was injected into the oocyte. Immediately following ooplasmic injection, the injection pipette was withdrawn quickly and the oocyte released from the holding pipette to reduce the intracytoplasmic pressure.

Putative zygotes were washed and incubated in NCSU-23 culture medium (108.73 mM NaCl, 4.78 mM KCl, 1.19 mM KH₂PO₄, 1.19 mM MgSO₄.7H₂O, 5.5 mM glucose, 1 mM glutamine, 7 mM taurine 5 mM hypotaurine, 25.07 mM NaHCO₃, 1.7 mM CaCl₂.2H₂O, 75 μg/mL Penicillin G, 50 μg/mL Streptomycin, 4 mg/mL BSA, pH 7.4) with mineral oil overlay at 39° C. in an atmosphere of 5% CO₂ for 48 h. After 48 h embryos that had undergone the first cell division were placed in fresh NCSU 23 culture media in the same manner as described above until blastocyst formation, 144 h post-IVF.

The results herein show that NAC amide supplementation yields superior results compared to controls.

TABLE 3

| | | | # of Embryos | # of Embryos |
|---|---|---|---|---|
| | | | reaching 2-cell | reaching |
| | Method of | Total # of | stage of | blastocyst stage |
| Treatment | Fertilization | Oocytes | development | of development |
| Control | IVF | 67 | 10 | 6 |
| NAC amide | IVF | 70 | 22 | 12 |
| Control | ICSI | 25 | 5 | 2 |
| NAC amide | ICSI | 24 | 12 | 5 |

As various changes can be made in the above methods and compositions without departing from the scope and spirit of the invention as described, it is intended that all subject matter contained in the above description, shown in the accompanying drawings, or defined in the appended claims be interpreted as illustrative, and not in a limiting sense.

1.-20. (canceled)

21. A method of in vitro fertilization, comprising cultivating oocytes and sperm in a medium supplemented to contain N-acetylcysteine amide (NAC amide), or a physiologically acceptable salt or ester thereof, wherein said oocytes are fertilized by said sperm in said NAC amide-supplemented medium.

22. The method according to claim 21, wherein the oocytes and sperm are from a non-human animal.

23. The method according to claim 21, wherein the oocytes and sperm are from a human.

24. The method according to claim 21, wherein the oocytes and sperm are from a transgenic animal.

25. The method according to claim 21, wherein the medium is supplemented to contain NAC amide in combination with GM-CSF.

26.-33. (canceled)

34. A cell culture medium for reducing or preventing oxidative stress in oocytes, sperm, or embryos cultured in vitro, comprising N-acetylcysteine amide (NAC amide), or a physiologically acceptable salt or ester thereof.

35. The medium of claim 34, wherein the oocytes, sperm, or embryos are from a non-human animal.

36. The medium of claim 34, wherein the oocytes, sperm, or embryos are from a human.

37. The medium of claim 34, wherein the oocytes, sperm, or embryos are from a transgenic animal.

38. The medium of claim 34, further comprising a balanced salt solution selected from the group consisting of M199, Synthetic Oviduct Fluid, PBS, BO, Test-yolk, Tyrode's, HBSS, Ham's F10, HTF, Menezo's B2, Menezo's B3, Ham's F12, DMEM, TALP, Earle's Buffered Salts, CZB, KSOM, BWW Medium, and emCare Media.

39. The medium of claim 38, wherein the cells are sperm, and the balanced salt solution is TALP or HTF.

40. The medium of claim 38, wherein the cells are embryos and the balanced salt solution is CZB.

41. The medium of claim 34, further comprising a buffering solution, one or more macromolecules, one or more additional free radical scavengers, one or more enzymes, one or more growth factors, one or more polymeric molecules, one or more antibiotics, one or more antimycotics, one or more hormones, or one or more proteins.

42. The medium of claim 34, wherein the cells are sperm, and wherein the medium optionally comprises sperm motility stimulants.

43. The medium of claim 42, wherein the sperm motility stimulants comprise caffeine, follicular fluid, calcium, oxytocin, kallikrein, prostaglandins, thymus extracts, pentoxyfilline, 2-deoxyadenosine, inositol, flavanoids, platelet activating factor, hypotaurine, chondroitin sulfate, or mercaptoethanol.

44. The medium of claim 34, wherein the medium further comprises GM-CSF.

45. A cell culture supplement for reducing or preventing oxidative stress in oocytes, sperm, or embryos cultured in vitro, comprising N-acetylcysteine amide (NAC amide), or a physiologically acceptable salt or ester thereof.

46.-56. (canceled)
